(19) United States
(12) Patent Application Publication
(10) Pub. No.: US 2016/0100950 A1
(43) Pub. Date: Apr. 14, 2016

(54) Total Knee Prostheses and Method for the Assembly Thereof

(71) Applicant: EUROS, La Ciotat (FR)

(72) Inventors: Yves Catonne, Paris (FR); José Gadea, Toulon (FR); Christophe Lecoq, Marseille (FR)

(73) Assignee: Euros, La Ciotat (FR)

(21) Appl. No.: 14/894,779

(22) PCT Filed: May 14, 2014
(86) PCT No.: PCT/FR2014/051122

§ 371 Date: Nov. 30, 2015

(30) Foreign Application Priority Data
May 30, 2013 (FR) 1354966

(51) Int. Cl. A61F 2/38

(52) U.S. Cl. CPC A61F 2/385; A61F 2/3886; A61F 2220/0041

(57) ABSTRACT

A method for assembling a total knee prosthesis is described, wherein portions of femoral and tibial implants, which are referred to as common portions and are each attachable in a femur and in a tibia of a knee, respectively, are assembled, and interchangeable portions are selected for adjustable assembly onto said common portions for each implant, respectively, depending on the type of prosthesis desired, some for a first posterior stabilization assembly configuration, and the others for a second rotary hinge assembly configuration.

The present invention relates to improvements to total knee prostheses and methods for the assembly thereof.

The field of the invention is that of prostheses specific to knees which effectively require particular techniques due to the complexity of the joint which should, in addition to the rotational mobility function thereof, particularly retain both lateral and antero-posterior stability while supporting the weight of the body.

As this joint which is subject to high stress is liable to sustain damage, it is then sometimes necessary to replace the joint completely by a prosthesis of which numerous models exist, the majority whereof have been the subject of numerous patent applications, of which, mention may be made for example of the prior applications by the present applicant, the company Euros, FR2721500 dated 22 Jun. 1994 and FR2678824 dated 11 Jul. 1991, or that by the company Merck Biomaterial France FR2780636 dated 6 Jul. 1998 or that by the company Dupuy FR2793676 dated 18 May 1999 or that among the most recent by M.Afriat FR2976176 dated 6 Jun. 2011.

Reference may advantageously be made thereto to understand the operation, positioning in relation to the bone extremities whereon they are fixed, the set of constituent components thereof, the various types and structures of various total knee prostheses without needing to explain everything in the present description: indeed, the non-essential elements for explaining the present invention will not be described herein, although some are represented in the figures but are thus not referenced in relation to the description.

Regardless of the total knee prosthesis used, it is certain that said prosthesis has a limited “lifetime”, very frequently shorter than that of the patient on whom it has been implanted, because, as mentioned above, the knee joint is subject to high stress and the contact surfaces and the moving parts and even the bone anchors may sustain damage: the prosthesis will thus need to be replaced one day.

However, it is known that surgeons are frequently faced with a problem in respect of ligament balancing either from the first operation for fitting the initial prosthesis or particularly during the second (and obviously subsequent) procedure to replace said prosthesis after the ablation of the first-line prosthetic implants in the perioperative stage. Indeed, during the ablation of the implants, in spite of the precautions required, it may arise that the lateral ligaments are damaged, or even detached from the bone insertion thereof.

Depending on how the operation proceeds, the surgeon then needs to change, in the event of failure of the lateral ligaments, from a posterior stabilised prosthesis to a lateral stabilised prosthesis corresponding to a hinge pivot system. In fact, this change requires envisaging, before the procedure and next to the operating table, the availability of both types of prostheses with two types of fitting ancillaries and, if the decision to change is made when the posterior stabilised prosthesis has already been fitted, the surgeon must remove same in order to fit the hinge pivot prosthesis.

The problem addressed and solved by the present invention is thus that of avoiding the drawbacks cited above in respect of the availability and change between two prostheses meeting different instability criteria during the same surgical procedure for implanting a total knee prosthesis and which are particularly encountered during revision surgery to replace a first prosthesis.

The solution to the problem addressed is a method for assembling a total knee prosthesis whereby:
parts of femoral and tibial implants, which are referred to as common parts and are each attachable in a femur and in a tibia of knee, respectively, are assembled,
interchangeable parts are selected for modular assembly onto said common parts for each implant, respectively, depending on the type of prosthesis desired, some for a first posterior stabilisation assembly configuration, and the others for a second lateral stabilisation or rotary or hinge pivot assembly configuration.

In the present description, the front and any so-called anterior portion of the prosthesis denote those corresponding to the same orientation as the front of the knee liable to receive this prosthesis, as for the posterior portions corresponding to the back of the knee, the lateral portions corresponding to the sides of the knee, the upper or top portions corresponding to the femur side orientation and the lower or bottom portions to the tibia side.

A further solution to the problem addressed is thus a total knee prosthesis consisting of at least:
a femoral implant comprising at least one anchoring pivot at its upper portion and attachable in a femur, a so-called anterior wall corresponding to the forward orientation of the prosthesis and two curved branches extending in the posterior direction from this anterior wall, forming condyles and separated by a hollow intercondylar space in the form of a groove, which groove defined by the inner edge of these condylar branches and the inner face of the anterior wall, and
a tibial implant comprising at least one anchoring pivot at its lower portion and attachable in a tibia, and a support plate suitable for supporting the loads of the condyles of the femoral implant,
and such that, according to the invention, these femoral and tibial implants are modular between a first posterior stabilisation assembly configuration and a second lateral stabilisation or rotary or hinge pivot assembly configuration, and each comprising a common part to both configurations, including at least the elements described above and specific to each implant, and a further interchangeable part adapted to each configuration and as described hereinafter by way of preferential examples of an embodiments.

These solutions may be considered to be improvements to total knee prostheses and address the problem stated as the modularity offered by the implant system according to the invention makes it possible with a single base and common part for the femoral implant, a single base and common part for the tibial implant, two specific parts for each base for each configuration, and a single fitting ancillary, to adapt to both scenarios to change in particular from a posterior stabilised prosthesis to a rotary hinge prosthesis.

The result is thus an improvement to total knee joint prostheses, for which the advantages mentioned above demonstrate the interest thereof and for which the description and the figures attached give examples of embodiments thereof.

Further embodiments are however possible within the scope of the present invention.

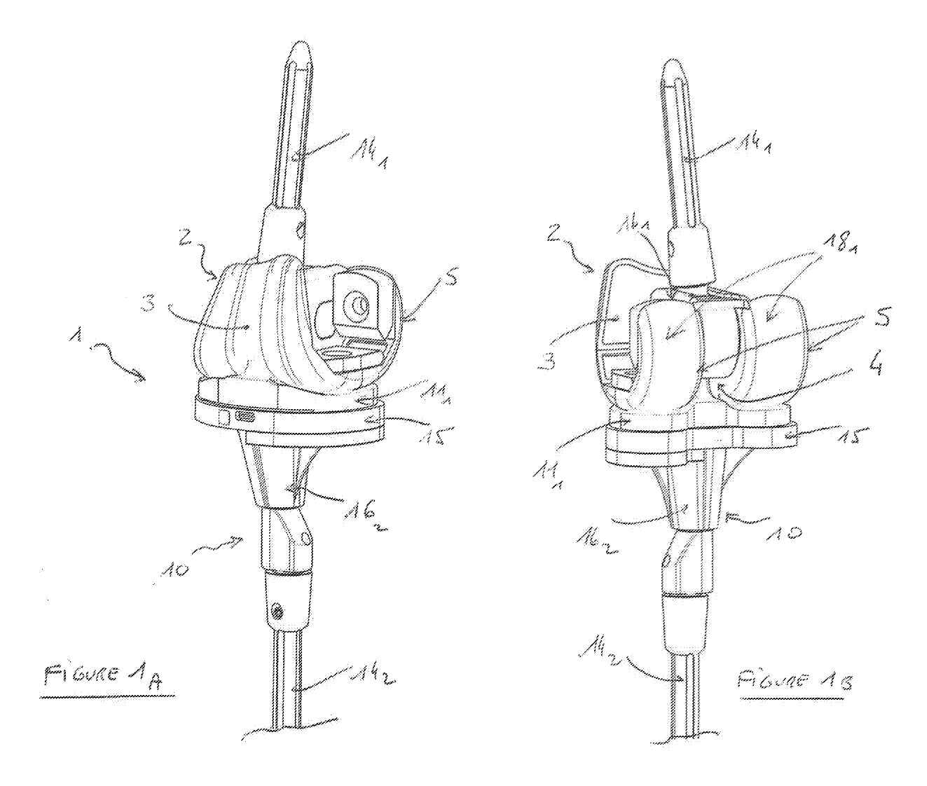

FIGS. 1A and 1B represent two perspective, front and rear three-quarter views of a total knee prosthesis according to the invention, fully assembled in the posterior stabilised configuration.

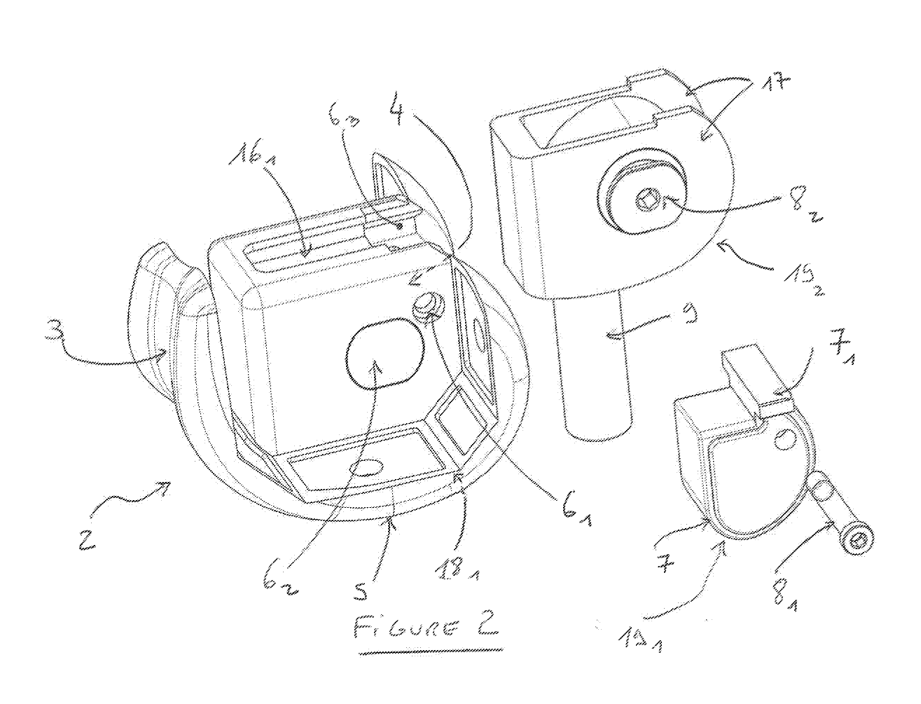

FIG. 2 represents a perspective top three-quarter view of a femoral implant according to the invention, without the anchoring pin thereof, with as a presentation of the choice of assembly two intercondylar <<cassettes>>, one in the posterior stabilised configuration and the other in the rotary hinge configuration.

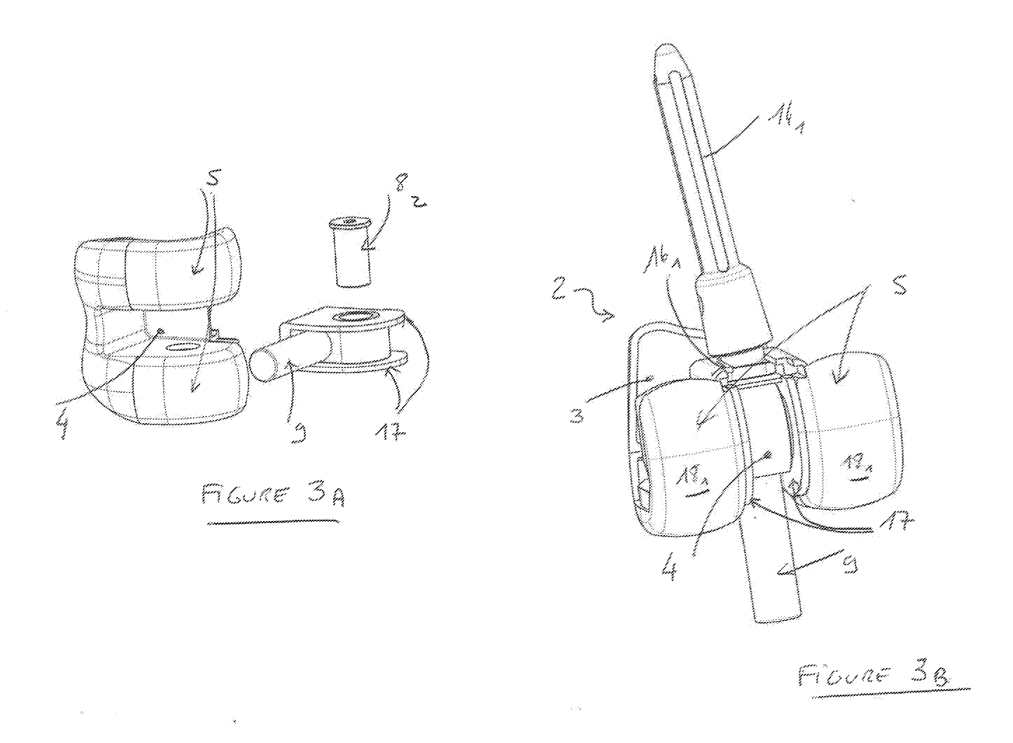

FIG. 3A represents the same femoral implant as FIG. 2 in a bottom view and before assembly of a <<cassette>> in the rotary hinge configuration.

FIG. 3B represents the same femoral implant as in FIGS. 2 and 3A in a rear perspective view, with the anchoring pin thereof and after assembling a <<cassette>> in the rotary hinge configuration.

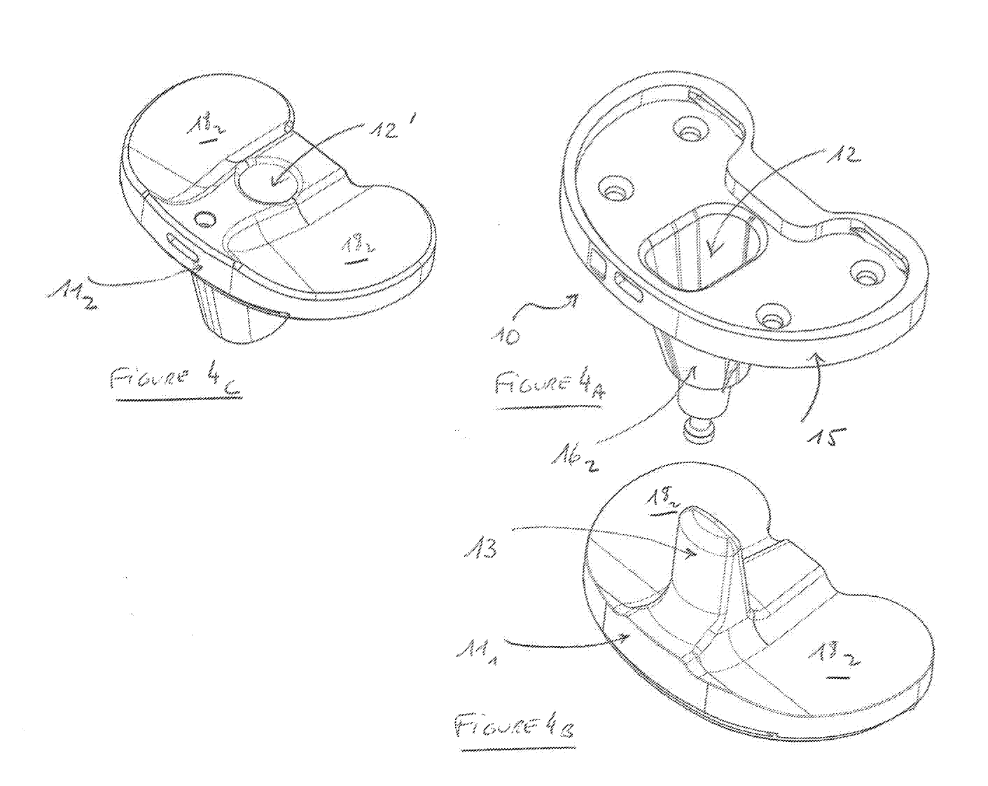

FIGS. 4A, 4B and 4C represent in a perspective three-quarter top view of a tibial implant according to the invention, with its common part according to FIG. 4A and the choice of assembly of its interchangeable part consisting of one of the two intermediate plates, one according to FIG. 4B for the posterior stabilised assembly and the other according to FIG. 4C for the rotary hinge assembly.

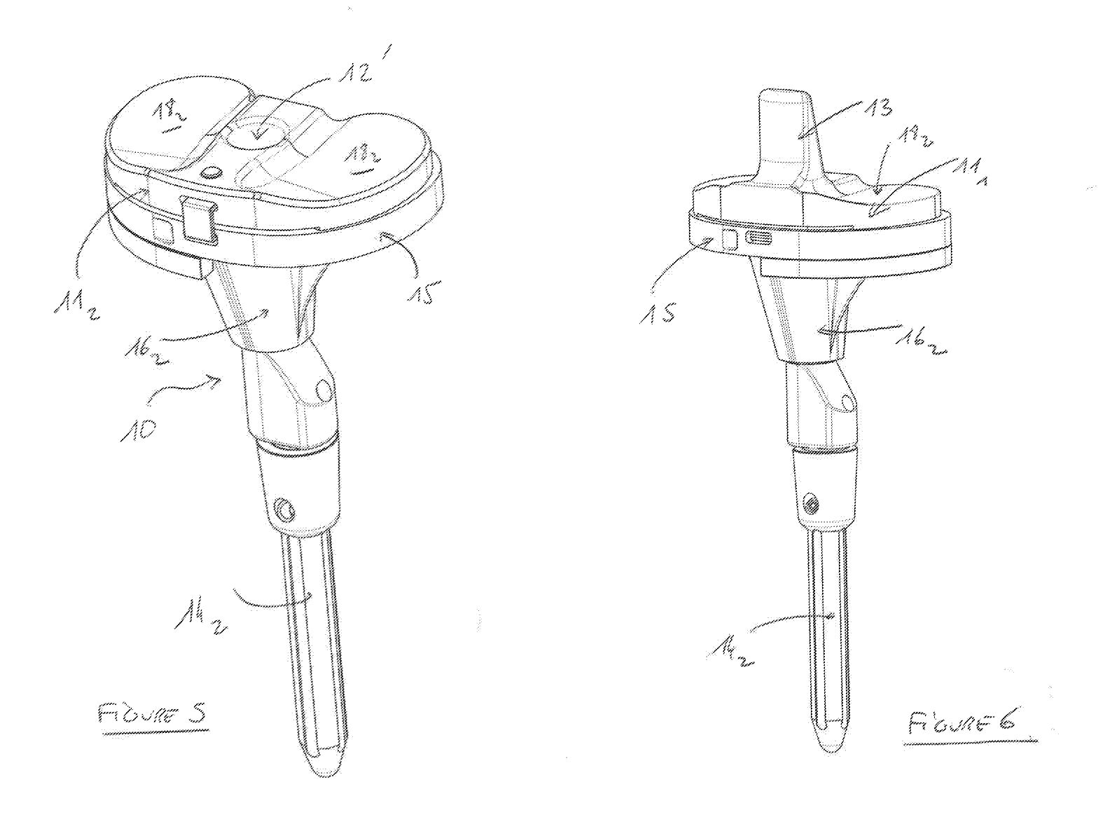

FIGS. 5 and 6 represent two perspective views, three-quarter top and front views of a tibial implant according to FIG. 4, fully assembled in the two configurations, posterior stabilised in FIG. 6 and rotary hinge in FIG. 5.

A total knee prosthesis 1 according to the invention consists of at least:
a femoral implant 2 comprising at least one anchoring pivot 14₁ at its upper portion and attachable in a femur, a so-called anterior wall 3 corresponding to the forward orientation of the prosthesis and two curved branches 5 extending in the posterior direction from this anterior wall 3, forming condyles and separated by a hollow intercondylar space 4 in the form of a groove, defined by the inner edge of these condylar branches 5 and the inner face of the anterior wall 3, and
a tibial implant 10 comprising at least one anchoring pivot 14₂ at its lower portion and attachable in a tibia, and a support plate 15 suitable for receiving an intermediate plate 11 supporting the loads of the condyles 5 of the femoral implant 2.

According to the invention, these femoral 2 and tibial 10 implants are modular between a first posterior stabilisation assembly configuration according to FIGS. 1, 4B and 6, and a second rotary hinge assembly configuration according to FIGS. 3, 4C and 5, and each comprising a common part to both configurations, including at least the elements described above specific to each implant, and a further interchangeable part adapted to each configuration and as described hereinafter by way of a preferential example of an embodiment.

According to FIG. 2, the femoral implant 2 comprises means for attaching at least one interchangeable part suitable for being assembled and disassembled from the common part of this femoral implant 2, and which is:
either a so-called first interchangeable part, consisting of at least one transversal element 7 perpendicular to said branches 5 and suitable for closing said intercondylar groove 4 on its posterior face and for forming a cage suitable, in this configuration, for receiving a posterior stabilisation pin 13 of the tibial implant 10 as represented in FIGS. 1, 4B and 6,
or a so-called second interchangeable part, consisting of at least one transversal shaft 8₂ perpendicular to said branches 5 and suitable for supporting and articulating a balance arm 9 extending from the intercondylar groove 4 beyond the condylar surfaces 5 and suitable in this configuration for being inserted and engaging in a hollow portion 12 of the base 16₂ of the anchoring pivot 14₂ of the tibial implant 10, as represented in FIGS. 3, 4A, 4C and 5.

The means for attaching the transversal element 7 and the balance arm 9 consist at least, on one hand, of orifices 6 formed in the inner walls of the branches 5 which, forming its edges, surround the groove 4 and, on the other hand, transversal shafts 8 passing through these orifices 6 and supporting either the transversal element 7 or the balance arm 9.

Preferably as represented in the same FIG. 2, this transversal element 7 is in the form of a first <<cassette>> 19₁ wherein the anterior face closes the cage suitable for receiving the posterior stabilisation pin 13 of the tibial implant 10 and the surface, extending this anterior face along the lower and rear portion of the <<cassette>> 19₁, is in a form suitable for rolling against the surface of the pin 13 during the rotation of the implants in relation to one another, and this <<cassette>> 19₁ comprises at its upper portion, to the rear of its anterior face and above its rear portion, a tenon 7₁ protruding from either side of the volume of the <<cassette>> inserted into the intercondylar groove 4, and which is suitable for engaging with and in a mortise 6₃ formed in the lateral walls of this groove.

Similarly, again according to this FIG. 2 but also as represented in FIG. 3A, the balance arm 9 pivoting about the transversal shaft 8₂ is mounted between two flanges 17 of a U-shaped part forming a second <<cassette>> 19₂ inserted into the intercondylar groove 4 and wherein the base of the U bears against the inner face of the anterior wall 3.

According to FIGS. 4, 5 and 6, the tibial implant 10 comprises means for attaching at least one interchangeable portion consisting of an intermediate plate 11 supporting the loads of the condyles 5 of the femoral implant 2 and which is suitable for being assembled and disassembled from the common part of this femoral implant 10; this common part comprises at least one anchoring pivot 14₂ at its lower portion and attachable in a tibia, and a support plate 15 suitable for receiving the interchangeable part which consists at least:
either according to FIG. 4B of a first intermediate plate 11₁ attached to the support plate 15 and comprising the posterior stabilisation pin 13 arranged between two bearing surfaces 18₂ suitable for receiving and engaging with the condylar surfaces 18₁ of the femoral implant 2, in the so-called posterior stabilisation assembly configuration
or according to FIG. 4C of a second intermediate plate 11₂ attached to the support plate 15 and comprising two bearing surfaces 18₂ suitable for receiving and engaging with the condylar surfaces 18₁ of the femoral implant 2 and between which opens, via an opening 12′, the hollow portion 12 of the base 16₂ of the tibial implant 10 for receiving and engaging with the balance arm 9 of the femoral implant 2 in the so-called lateral stabilisation or rotary or hinge pivot assembly configuration.

As such, the method for assembling a total knee prosthesis 1, as represented in the attached figures and according to the invention, is such that:
femoral and tibial implant parts, so-called common respectively for each implant, are assembled, and comprising at least for the femoral implant 2 an anchoring pivot 14₁ at its upper portion, a so-called anterior wall 3 corresponding to the forward orientation of the prosthesis and two curved branches 5 extending in the posterior direction from this anterior wall 3, forming condyles and separated by a hollow intercondylar space 4 in the form of a groove, defined by the inner edge of these condylar branches 5 and the inner face of the anterior wall 3, and comprising at least for the tibial implant 10 an anchoring pivot 14₂ at its lower portion and a support plate 15 suitable for supporting the load of the condyles 5 of the femoral implant 2, and
for the posterior stabilisation assembly, for the femoral implant 2, a first interchangeable part as described above and represented in FIG. 2 and for the tibial implant a first interchangeable part as described above and represented in FIG. 4B are selected,
and for the rotary hinge assembly, the first interchangeable parts described above are selected or replaced, for the femoral implant 2, by a second interchangeable part as described above and represented in FIGS. 2 and 3A, and, for the tibial implant, by a second interchangeable part as described above and represented in FIG. 4C.

1.-9. (canceled)

10. A total knee prosthesis comprising:
a femoral implant and a tibial implant, each being modular between a first posterior stabilization assembly configuration and a second rotary hinge assembly configuration;
the femoral implant comprising a femoral interchangeable part adapted to each assembly configuration and a femoral common part to both assembly configurations that includes at least one anchoring pivot at an upper portion and attachable in a femur, an anterior wall corresponding to a forward orientation of the prosthesis and two curved branches extending in a posterior direction from the anterior wall, forming condyles and separated by a hollow intercondylar space in a form of a groove that is defined by an inner edge of the condylar branches and an inner face of the anterior wall;
the tibial implant comprising a tibial interchangeable part adapted to each assembly configuration, and a tibial common part to both assembly configurations that includes at least one anchoring pivot at a lower portion and attachable in a tibia, and a support plate configured to support a load of the condyles of the femoral implant;
wherein the femoral implant includes at least one or both of:
at least one transversal element to removably attach at least a first femoral interchangeable part to the femoral common part, the transversal element being perpendicular to the branches and configured to close the intercondylar groove on its posterior face and to form a cage to receive a posterior stabilization pin of the tibial implant, and
at least one transversal shaft to removably attach a second femoral interchangeable part to the femoral common part, the transversal shaft being perpendicular to the branches and configured to support and articulate a balance arm extending from the intercondylar groove beyond the condylar surfaces and suitable in this configuration for being inserted and engaging in a hollow portion of a base of the anchoring pivot of the tibial implant.

11. The total knee prosthesis according to claim 10 wherein the femoral implant further comprises orifices formed in inner walls of the branches which, forming its edges, surround the groove and transversal shafts passing through the orifices and supporting either the transversal element or the balance arm.

12. The total knee prosthesis according to claim 10 wherein
when the femoral implant includes the at least one transversal element, the tibial implant includes a first intermediate plate to removably attach a first interchangeable part to the tibial common part, the first intermediate plate being attached to the support plate and comprising the posterior stabilization pin arranged between two bearing surfaces configured to receive and engage with the condylar surfaces of the femoral implant, and
when the femoral implant includes the at least one transversal shaft, the tibial implant includes a second intermediate plate to removably attach a second interchangeable part to the tibial common part, the second intermediate part being attached to the support plate and comprising two bearing surfaces configured to receive and engage with the condylar surfaces of the femoral implant and between which opens, via an opening, the hollow portion of the base of the tibial implant.

13. The total knee prosthesis according to claim 10, wherein the transversal element is in the form of a cassette having an anterior face that closes the cage and a surface that extends along a lower and rear portion of the cassette and is configured to roll against a surface of the posterior stabilization pin during the rotation of the implants in relation to one another, wherein the cassette comprises at an upper portion, to a rear of the anterior face and above a rear portion, a tenon that protrudes from either side of a volume of the cassette inserted into the intercondylar groove, the tenon configured to engage with and in a mortise formed in lateral walls of the intercondylar groove.

14. The total knee prosthesis according to claim 11, wherein the transversal element is in the form of a cassette having an anterior face that closes the cage and a surface that extends along a lower and rear portion of the cassette and is configured to roll against a surface of the posterior stabilization pin during the rotation of the implants in relation to one another, wherein the cassette comprises at an upper portion, to a rear of the anterior face and above a rear portion, a tenon that protrudes from either side of a volume of the cassette inserted into the intercondylar groove, the tenon configured to engage with and in a mortise formed in lateral walls of the intercondylar groove.

15. The total knee prosthesis according to claim 10, wherein the balance arm is mounted between two flanges of a U-shaped part forming a cassette inserted into the intercondylar groove and wherein a base of the U-shaped part bears against the inner face of the anterior wall.

16. The total knee prosthesis according to claim 11, wherein the balance arm is mounted between two flanges of a U-shaped part forming a cassette inserted into the intercondylar groove and wherein a base of the U-shaped part bears against the inner face of the anterior wall.

17. A method for assembling a total knee prosthesis according to claim 13 wherein:
assembling the common femoral and tibial implant parts;
for the posterior stabilization assembly configuration, assembling the femoral interchangeable part with the common femoral implant part by providing the at least one transversal element and by inserting the posterior stabilization pin of the tibial implant into the cage and assembling the tibial interchangeable part with the common tibial implant part by attaching the first intermediate plate to the support plate and arranging the posterior stabilization pin between the two bearing surfaces configured to receive and engage with the condylar surfaces of the femoral implant; or
for the rotary hinge assembly configuration, assembling the femoral interchangeable part with the common femoral implant part by providing the at least one transversal shaft and inserting the balance arm in the hollow portion of the base of the anchoring pivot of the tibial implant and assembling the tibial interchangeable part with the common tibial implant part by attaching the second intermediate plate to the support plate and engaging the condylar surfaces of the femoral implant.

18. A method for the assembly of a total knee prosthesis comprising:
assembling femoral and tibial implant parts, each having respective common parts and each respectively attachable in a femur and a tibia of a knee;
selecting a respective interchangeable part for modular assembly onto the respective common parts for each implant and, depending on a posterior stabilization assembly configuration or a rotary hinge assembly configuration; and,
assembling a selected interchangeable part with a femoral implant common part and assembling a selected interchangeable part with a tibial implant common part.
